(19) United States
(12) Patent Application Publication
(10) Pub. No.: US 2019/0010195 A1
(43) Pub. Date: Jan. 10, 2019

(54) RECOMBINANT SPIDER SILK PROTEIN COATINGS AND ADHESIVES FOR MEDICAL DEVICES

(71) Applicants: Randolph V. Lewis, Nibley, UT (US); Justin A. Jones, Nibley, UT (US); Thomas Ian Harris, Logan, UT (US); Danielle Gaztambide, Logan, UT (US); Breton A. Day, Highland, UT (US)

(72) Inventors: Randolph V. Lewis, Nibley, UT (US); Justin A. Jones, Nibley, UT (US); Thomas Ian Harris, Logan, UT (US); Danielle Gaztambide, Logan, UT (US); Breton A. Day, Highland, UT (US)

(73) Assignee: Utah State University, Logan, UT (US)

(21) Appl. No.: 15/417,106

(22) Filed: Jan. 26, 2017

Related U.S. Application Data
(60) Provisional application No. 62/287,311, filed on Jan. 26, 2016.

(51) Int. Cl. C07K 14/435; A61L 27/22; A61L 27/54

(52) U.S. Cl. CPC C07K 14/43518; A61L 2420/02; A61L 27/54; A61L 27/227

(57) ABSTRACT

Medical devices made with coatings made from recombinant spider silk proteins are disclosed. Methods to make the devices are also disclosed. Methods for adhering objects to one another using recombinant spider silk proteins are also disclosed.

### RELATED APPLICATIONS

This application claims priority to U.S. Provisional Application No. 62/287,311, filed on Jan. 26, 2016, the entirety of which is hereby incorporated by reference.

### TECHNICAL FIELD

The present disclosure relates to coatings and adhesives for surfaces used in medical devices with synthetic spider silk protein compositions and methods of their preparation.

### BACKGROUND

Plastic and metallic components are frequently used in medical devices. The material used for those components are often chosen with one or more material properties required for the function of the component, such as high flexural modulus, high tensile and compressive strength, or capacity to be shaped into useful forms. Those component materials, however, are frequently water-insoluble and hydrophobic, so that they are poorly wetted by water. Thus, water tends to form beads on the surface, or the material can sometimes act as a site for inflammatory responses or other undesirable biological outcomes. In addition, body implants, such as orthopedic joints and other bone replacements, often present problems with subnormal lubrication as the body recovers from the trauma of disease and injury and their corrective treatment.

A number of agents have been used to ameliorate the unwanted side effects arising from biologically incompatible materials. Many polymer-based agents, however, are increasingly the suspected cause of detrimental effects when they slough off of a coated device in a patient. In addition, some coatings require a long and multi-step process to be applied to a substrate. Moreover, these coatings have shortcomings and fail to provide lasting or effective relief to the undesirable side effects of the incompatible material. There remains, therefore, a need to provide alternative materials for coating medical device components that impart better lubricity, wettability, and biocompatibility. There also remains a need to provide alternative materials for adhering components in such devices together that are both biocompatible as well as satisfactory for the desired adhesion properties.

### SUMMARY

In one aspect, a method of coating a medical device is disclosed which includes solubilizing one or more recombinant spider silk proteins in an aqueous solution; providing a medical device having a substrate surface; applying the solubilized one or more recombinant spider silk proteins in the aqueous solution to the substrate surface; and drying the device surface.

In another aspect, a method of adhering two objects is disclosed which includes solubilizing one or more recombinant spider silk proteins in an aqueous solution; providing a first object having a substrate surface; applying the solubilized one or more recombinant spider silk proteins in the aqueous solution to the substrate surface; providing a second objecting having a target surface; and contacting the substrate surface and the target surface to adhere the first and second objects together. In some embodiments, the method includes removing water (drying) from between the substrate and target surfaces.

In some embodiments, the solubilizing step further includes mixing the one or more recombinant spider silk proteins with water to form a mixture in a sealed container; and heating the mixture. In some embodiments, the heating is performed with microwave irradiation.

In some embodiments, the application of the solubilized recombinant spider silk is by an aerosolizing sprayer. In some embodiments, the application of the solubilized recombinant spider silk in is performed by dipping the substrate surface in the aqueous solution.

In some embodiments, the method also includes applying a base-layer coating to the substrate surface prior to applying additional layers of solubilized recombinant spider silk.

In some embodiments, the methods include the additional step of adding an additive to the aqueous solution. In some embodiments, the additive is a bicarbonate salt. In some embodiments, additive decreases drying time. In some embodiments, the additive is an organic alcohol or organic acid.

In some embodiments, the substrate surface is selected from a cellulosic polymer, a silicone polymer, a plastic polymer, and a metal. In some embodiments, the target surface of the second object is selected from a cellulosic polymer, a silicone polymer, a plastic polymer, and a metal.

In some embodiments, the one or more recombinant spider silk proteins is selected from the group consisting of: M4, M5, MaSp1, an MaSp1 analogue, MaSp2, an MaSp2 analogue, FLYS, FLAS, AcSp (aciniform silk protein), AgSp2 (aggregate gland silk protein 2), and combinations thereof.

In some embodiments, the methods include adding a medicinal agent. In some embodiments, the medicinal agent is selected from an antimicrobial agent, an anti-clotting agent, and a therapeutic agent. In some embodiments, the medicinal agent is an antibacterial agent. In some embodiments, the medicinal agent is an antifungal agent. In some embodiments, the medicinal agent is an anti-clotting agent. In some embodiments, the anti-clotting agent is heparin. In some embodiments, the medicinal agent is a therapeutic agent.

In another aspect, a medical device is prepared from any one of the aforementioned methods. In some embodiments, the the medical device is selected from a catheter, a splint, a bandage, a drain tube, and an implant.

### BRIEF DESCRIPTION OF THE DRAWINGS

FIG. 1 illustrates exemplary surface friction data from embodiments, specifically comparative status and kinetic friction profiles for uncoated (UC) and coated (C) that reduce the coefficients of friction by 65%.

FIGS. 2A and 2B illustrate exemplary water contact angles from embodiments.

FIG. 2A illustrates an uncoated silicone surface with an angle of 112 degrees. FIG. 2B illustrates spider silk coated silicone with an average angle of 57 degrees.

FIGS. 3A-3H illustrate exemplary functionalized coatings from embodiments and their various impacts on microbial growth. Each of FIGS. 3A-3G illustrate inhibition zones where silk was functionalized with the following antibacterials (listed in order from the top then clockwise): chloramphenicol, tetracycline, ampicillin, gentamicin, kanamycin, silk control, and center: no coating. FIG. 3A illustrates Staphylococcus aureus with functionalized spider silk treated as set forth above. FIG. 3B illustrates Serratia marcescens with functionalized spider silk treated as set forth above. FIG. 3C illustrates Escherichia coli with functionalized spider silk treated as set forth above. FIG. 3D illustrates Escherichia coli with stainless steel with functionalized spider silk treated as set forth above. FIG. 3E illustrates Pseudomonas aeruginosa with functionalized spider silk treated as set forth above. FIG. 3F illustrates Pseudomonas aeruginosa under UV with functionalized spider silk treated as set forth above. FIG. 3G illustrates Escherichia coli with silicone with functionalized spider silk treated as set forth above. FIG. 3H illustrates Escherichia coli and polyurethane.

FIGS. 4A and 4B illustrate exemplary functionalized coatings from embodiments and their various impacts on microbial growth. FIG. 4A illustrates Candida albicans and silicone where the spider silk was functionalized with azole and experimental antifungals.

FIG. 4B illustrates Candida albicans and silicone where the spider silk was functionalized with antifungals.

FIG. 5 illustrates exemplary adhesives and their max stress, specifically, from left to right: Elmer's Wood Glue, Gorilla Glue, Masp1/Masp2, and FLYS3.

FIG. 6 illustrates exemplary adhesives and their max stress, specifically, from left to right: Elmer's Glue, Masp1/Masp2, Super Glue, Masp1/Masp2, FLYS3, Masp1/Masp2, Masp1/Masp2 Bicarbonate, Masp1/Masp2, and FLY S3.

### DETAILED DESCRIPTION

The present disclosure covers methods, compositions, and reagents for making medical devices with coatings or adhesives from synthetic spider silk protein compositions. The synthetic spider silk proteins are sometimes referred to as of regenerated spider silk proteins (rSSP) or recombinant spider silk proteins.

In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

rSSP's are conventionally dissolved in a very harsh organic solvent, 1,1,1,3,3,3-hexafluoroisopropanol (HFIP), to create “dopes” that can be used to create fibers, films, gels and foams. HFIP has been widely used and accepted as it is the only solvent that: (1) dissolves rSSP's at high concentrations (30% w/v) providing uniformity between various groups testing data, (2) is sufficiently volatile and miscible to be removed rapidly from the forming fiber, (3) leaves little to no residue behind that could interfere with fiber formation. In addition, rSSP's generally are insoluble in aqueous solutions after purification, necessitating an organic solvent that meets the criteria outlined in 1-3. There are significant problems, however, with solvating rSSP's in HFIP or other organic solvents.

Dissolving rSSP in HFIP and then using pressure to extrude a dope into a coagulation bath does not allow the appropriate structures to form (notably β-sheets) to an extent that the fibers or films have to be post-spin processed multiple times to achieve protein structures that result in appreciable mechanical properties. See Lazaris et al., Spider Silk Fibers Spun from Soluble Recombinant Silk Produced in Mammalian Cells, Science 295, 472-476 (2002) (hereinafter “Lazaris”); and Teule et al., Modifications of spider silk sequences in an attempt to control the mechanical properties of the synthetic fibers, J. Mater Sci, 42, 8974-8985 (2007) (hereinafter “Teule”).

The cost of purchase and subsequent disposal of HFIP may be restrictive or prohibitive in an industrial setting of mass production. HFIP's cost of purchase is roughly $1,000/100 ml's of HFIP and 100 ml's of HFIP would likely be capable of solvating 20-30 g's of rSSP (20-30% w/v). Water is cheap even in its purest form. Per the MSDS published on Sigma Aldrich's web-site, disposal of HFIP requires; “Dissolve or mix the material with a combustible solvent and burn in a chemical incinerator equipped with an afterburner and scrubber,” a process that inherently has costs associated with it. Excess water can be evaporated or recycled and reused. Worker safety when utilizing such harsh, volatile solvents is also a consideration. As refleced the product material safety data sheet (MSDS): “Material is extremely destructive to tissue of the mucous membranes and upper respiratory tract, eyes, and skin. Cough, Shortness of breath, Headache, Nausea” (SIC). Water has no such requirements. Finally, the process of producing rSSP products could not be considered “green” using HFIP.

rSSP's are largely insoluble in water. There are a few notable exceptions: Teule describes a series of proteins (Y₁S₈ and A₂S₈) that were produced in bacteria and purified via Ni⁺⁺ chromatography. Short fibers were pulled straight from the eluted, pure rSSP fraction. See Teule. Lazaris describes ADF-3 (Araneus diadematus MaSp1) produced in mammalian cell culture. Water soluble ADF-3 was concentrated in the presence of glycine and extruded into a coagulation bath. A final example is a series of recombinant aciniform-like synthetic proteins that were able to be spun from an aqueous solution very similar to Teule 2007 (Xu 2012). See Xu et al., Recombinant Minimalist Spider Wrapping Silk Proteins Capable of Native-Like Fiber Formation. PloS-One 7(11): e50227. Doi: 10.1371/journal.pone.0050227 (2012). However, outside of this small sub-set of rSSP's, water solubility is elusive. The majority of these proteins were much smaller than the natural proteins and thus are unlikely to make mechanically useful fibers.

U.S. Patent Application Publication No. 2011/0230911 filed by Amsilk utilizes a top down approach: genetic manipulations and expression system manipulations to try and create water soluble silk proteins. However, such processes are costly both in time to create the manipulations/cell lines and also in that the proteins appear to be expressed in mammalian cell cultures. The culture conditions for such cell lines are not only personnel and time intensive but also the ingredients and equipment are substantially more expensive than the more traditional bacterial expression systems. In addition, such methods are limiting as there are not that many iterations of various spider silk repeats that can be expressed in this manner that will result in a water soluble protein that has appreciable mechanical properties.

To address these and other challenges, aqueous dopes can be prepared as disclosed in U.S. application Ser. No. 14/459,244, incorporated herein by reference, which identifies some methods for solubilizing rSSP's in aqueous solutions and then creating resulting spider silk compositions therefrom.

In general, methods of preparing aqueous dopes of rSSP may include the following steps: mixing rSSP, water, and optional additives; optionally sonicating the mixture; microwaving the mixture; and optionally centrifuging the mixture to solubilize the rSSP's.

rSSP and water are combined to create a doping mixture of greater than about 2% w/v (e.g. 0.02 g SSpS: 1 mL H₂O). In embodiments, the w/v does not typically exceed 50%. However, any percentage of less than 50% may be used.

Suitable rSSP's include: MaSp1 (as described in U.S. Pat. Nos. 7,521,228 and 5,989,894), MaSp2 (as described in U.S. Pat. Nos. 7,521,228 and 5,989,894), MiSp1 (as described in U.S. Pat. Nos. 5,733,771 and 5,756,677), MiSp2 (as described in U.S. Pat. Nos. 5,733,771 and 5,756,677), Flagelliform (as described in U.S. Pat. No. 5,994,099), chimeric rSSP's (as described in U.S. Pat. No. 7,723,109), Pyriform, aciniform, tubuliform, aggregate gland silk proteins, and AdF-3 and AdF-4 from araneus diadematus. Each of the above referenced patents is herein incorporated by reference in its entirety.

In one aspect, a medical device is disclosed. The device can be any medical device contemporary for use in treating patients and animals that has a surface that would benefit from being coated or that has parts that can be adhered to one another.

The devices are prepared by solubilizing one or more recombinant spider silk proteins in an aqueous solution. A substrate surface is then coated with the solubilized recombinant spider silk proteins. In embodiments where the coating is desired, the coated surface is then dried.

In embodiments where the device has two surfaces that need to adhere to one another, the method also includes providing a second object having a target surface that, when contacted with the substrate surface of a first object and solubilized, adheres with a first object. The adhesion can then be dried.

Exemplary medical devices can be made of a variety of materials, including components made of differing materials. For example, the device or component material and, therefore, the substrate surface can be made up of a cellulosic polymer, a silicone polymer, a plastic polymer, and a metal and combinations or segments of the same. The second object or component can be made of the same material or a different material.

In some embodiments, the plastic polymer is selected from polyurethane (PU), polystyrene (PS), polycarbonate (PC), polyethylene (PE), polypropylene (PP), expanded Teflon (ePT1-B), rubber, and latex. In some embodiments, the silicone polymer is silicone. In some embodiments, the metal is selected from stainless steel, titanium, and aluminum. In some embodiments, the cellulosic polymer is wood.

Exemplary devices include a catheter, a splint, a bandage, a drain tube, and an implant. Orthopedic devices can also include coatings or adhesions as described herein.

### Dope Additives

Various optional additives may be optionally added to the mixture. Suitable additives include compositions that contribute to the solubility of the rSSP in the solution. Some additives break or weaken disulfide bonds, thereby increasing the solubility of rSSP's. Other additives also serve to prevent hydrogel formation after the completion of the microwave heating step. If the solution forms a hydrogel quickly and the desired end product is not a gel, then additives capable of delaying or inhibiting such a formation may be desirable. In some embodiments, multiple additives may be added to achieve desired end products.

For example, to combat hydrogel formation, various additives may be added to the suspension of rSSP and water prior to microwaving the suspension. In some embodiments, acid, base, free amino acids, surfactants, or combinations thereof may be employed to combat hydrogel formation. For example, additions of acid (formic acid and acetic acid alone or together at 0.1% to 10% v/v), base (ammonium hydroxide at 0.1% to 10% v/v), free amino acids (L-Arginine and L-Glutamic Acid at 1 to 100 mM) as well as a variety of surfactants (Triton X-100 at 0.1% v/v) may be used. The additions of these various chemicals not only aid the solubility of rSSP when microwaved but in certain combinations also delay the solution from turning into a hydrogel long enough for the solution to be applied as a coating or adhesive.

Exemplary additives also include compositions capable of breaking or weakening disulfide bonds, such as β-mercaptoethanol or dithiothreitol may be added to reduce bonds and increase solubility. Suitable amounts of such additives may include from about 0.1 to about 5% (v/v). In embodiments where the rSSP does not contain cysteine, the use of such additives may be unnecessary. In some embodiments employing major ampulate silk proteins 1 and 2 (MaSp1 and MaSp2, respectfully), disulfide bonds (cysteine) are present in the C-terminus of the non-repetitive regions of MaSp1 and MaSp2. These proteins are described in U.S. Pat. Nos. 7,521,228 and 5,989,894, the entirety of which is herein incorporated by reference. In addition, the C-term is present in various goat-derived spider silk proteins M4, M5 and M55 proteins, which are described in U.S. Patent Application Publication No. 20010042255 A1, the entirety of which is incorporated by reference in its entirety. In some embodiments, formic acid and/or acetic acid may be included in as little as 0.3% (v/v) but even lower amounts (0.1% v/v) are possible. Additionally, it is possible to solubilize rSSP without using any additives.

Exemplary additives are set forth in Table 1 (below), where dope formulations prepared according to the methods described herein.

TABLE 1
Additives

| | | 3 | 4 | | 6 |
|---|---|---|---|---|---|
| 1 | 2 | Free Amino | Disulfide | 5 | Drying |
| Acid | Base | Acids | Reduction | Other | Agent |
| Acetic | Ammonium | Arginine | β- | Triton X-100 | Methanol |
| | Hydroxide | | mercaptoethanol | | |
| Formic | Sodium | Glutamic | Dithiothreitol | Glutaraldahyde | Ethanol |
| | Hydroxide | Acid | | | |
| Trifluoroacetic | | Histidine | | Calcium | Propanol |
| acid | | | | | |
| Other Organic | | Glycine | | Potassium | |
| Acids | | | | | |
| Propionic Acid | | Imidazole | | Other | |
| | | | | Surfactants | |
| | | Other Free | | Other Ions | |
| | | Amino | | | |
| | | Acids | | | |
| | | | | L-DOPA | |

In some embodiments, aqueous spin dopes omit additives. In some embodiments, the aqueous spin dope includes imidazole. In some embodiments, the aqueous spin dope includes propionic acid.

To formulate an aqueous solution of rSSP, additives can be chosen from any of the 6 columns or other additives described herein. For instance one or a combination of acids can be chosen from column 1 and combined with one or combinations of free amino acids from column 3, as well as disulfide reducing compounds from column 4 and “Other” additives as required or desired by the particular protein or application. Generally, it would not be useful to include both an acid from column 1 with a base from column 2. However, a base from column 2 can be combined with additives from columns 3-4.

In some embodiments, the additive reduces the drying time of the aqueous dope after it is applied to a surface. Examples of such additives include those listed in column 6 of Table 1. Other alcohols may be used so long as they increase the rate of evaporation relative to distilled water.

In some embodiments, the aqueous dopes may be augmented with bicarbonate solution. The bicarbonate solution may be of from 0.001-1 M bicarbonate. The bicarbonate solution may be from 0.01 to 1 M bicarbonate. The bicarbonate solution may be 0.1 to 1.0 M bicarbonate. The bicarbonate may be from ammonium, alkaline, and alkaline earth bicarbonate, e.g. sodium bicarbonate, potassium bicarbonate, calcium bicarbonate. In some embodiments, the bicarbonate is from ammonium bicarbonate.

In some embodiments, the doping solution includes one or more medicinal agents as additives. The medicinal agent is selected from an antimicrobial agent, an anti-clotting agent, and a therapeutic agent.

Representative antimicrobial agents include those which kill microorganisms or inhibit their growth. Examples include antibacterial and antifungal agents.

Examples of antibacterial agents include: ceftobiprole, ceftaroline, clindamycin, calbavancin, daptomycin, linezolid, mupirocin, oritavancin, tedizolid, telavancin, tigecycline, and vancomycin. Other examples of antibacterial agents include: aminoglycosides, carbapenems, ceftazidime, cefepime, fluoroquinolones, piperacillin, ticarcillin. Still other examples of antibacterial agents include: amikacin, gentamicin, kanamycin, neomycin, netilmicin, tobramycin, paromomycin, streptomycin, spectinomycin. Still other examples of antibacterial agents include: geldanamycin, herbimycin, and rifaximin. Still other examples of antibacterial agents include: loracarbef. Still other examples of antibacterial agents include: ertapenem, doripenem, imipenem/cilastatin, meropenem. Still other examples of antibacterial agents include: cefadroxil, cefazolin, cefalotin, cephalexin. Still other examples of antibacterial agents include: cefaclor, cefamandole, cefoxtin, cefprozil, cefuroxime, cefixime, cefdinir, cefditoren, cefoperazone, cefotaxime, cefpdoxime, ceftazidime, ceftibuten, ceftizoxime, and ceftriaxone. Still other examples of antibacterial agents include cefpime. Still other examples of antibacterial agents include: ceftaroline fosamil and ceftobiprole. Still other examples of antibacterial agents include: teicoplanin, vancomycin, telavancin, dabavancin, and oritavancin. Still other examples of antibacterial agents include: clindamycin and lincomycin. Still other examples of antibacterial agents include daptomycin. Still other examples of antibacterial agents include: azithromycin, clarithromycin, dithromycin, erythromycin, roxithromycin, troleandomycin, telithromycin, and spiramycin. Still other examples of antibacterial agents include aztreonam. Still other examples of antibacterial agents include: furazolidone and nitrofurantoin. Still other examples of antibacterial agents include: linezolid, posizolid, radezolid, torezolid. Still other examples of antibacterial agents include: amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin. Still other examples of antibacterial agents include: bacitracin, colistin, and polymyxin B. Still other examples of antibacterial agents include: ciprofloxacin, enoxacin, gatifloxacin, levofloxacin, lomefloxacin, moxiflacacin, nalidxic acid, norflacin, ofloxacin, trovafloxacin, grepafloxacin, sparfloxacin, temafloxacin, mafenide, sulfacetamide, sulfadizazine, silver sulfadazine, sulfadimethoxine, sulfamethizole, sulfamethoxazole, sulfanilamide, sulfasalazine, sulfisoxacole, trimethoprim-sulfamethoxazole, sulfonamidochrysoidine. Still other examples of antibacterial agents include: demeclocycline, doxycycline, minocycline, oxytetracycline, tetracycline. Still other examples of antibacterial agents include: clofazimine, dapsone, capreomycin, cycloserine, ethambutol, ethionamide, isoniazid, pyrazinamide, rifampicin, rifabutin, rifapentine, streptomycin. Still other examples of antibacterial agents include: arsphenamine, chloramphenicol, fosformycin, fusidic acid, metronidazole, mupirocin, platensimycin, quinupristin/dalfopristin, thiampenicol, tigecycline, tinidazole, and trimethorprim. Still other examples of antibacterial agents include combinations of the foregoing.

In some embodiments, the antimicrobial agent is an anti-fungal agent. The anti-fungal agent may be selected from amphotericin B, candicidin, filipin, hamycin, natamycin, nystatin, and rimocidin. The anti-fungal agent may be selected from bifonazole, butoconazole, clotrimazole, econazole, fenticonazole, isoconazole, ketoconazole, luliconazole, miconazole, omoconazole, oxiconazole, sertaconazole, sulconazole, tioconazole. The anti-fungal agent may be selected from albaconazole, efinaconazole, epoxiconazole, fluconazole, isavuconazole, itraconazole, posaconazole, propiconazole, ravuconazole, terconazole, and voriconazole.

The anti-fungal agent may be selected from abafungin, amorolfin, butenafine, naftifine, terbinafine, echinocandins, anidulafungin, caspofungin, micafungin. The anti-fungal agent may be selected from benzoic acid, ciclopirox, flucytosine, griseofulvin, haloprogin, tolnaftate, undecylenic acid, crystal violet.

In some embodiments, the medicinal agent is an anti-clotting agent. In some embodiments, the anti-clotting agent is a coumarin (vitamin K antagonists) such as warfarin, acenocoumarol, phenprocoumon, atrometnin, and phenindone. In some embodiments, the anti-clotting agent is heparin. In some embodiments, the anti-clotting agent is a synthetic pentasaccharide inhibitor of factor Xa such as fondaparinux and idraparinux. In some embodiments, the anti-clotting agent is a direct factor Xa inhibitor such as rivaroxaban, apixaban, edoxaban, betrixaban, darexaban, letaxaban, and eribaxaban. In some embodiments, the anti-clotting agent is a direct thrombin inhibitor such as hirudin, lepirudin, bivalirudin, argatroban, dabigatran. In some embodiments, the anti-clotting agent is an antithrombin protein including antithrombin and recombinant antithrombin. In some embodiments, the anti-clotting agent is aspirin.

In some embodiments, the medicinal agent is a therapeutic agent. Examples of therapeutic agents include a variety of agents including those which have an intended therapeutic outcome for a patient need of a necessary treatment. In some embodiments, the therapeutic agent is a growth factor such as a protein or steroid hormone. In some embodiments, the growth factor is selected from adrenomedullin (AM), angiopoietin (Ang), autocrine motility factor, bone morphogenetic proteins (BMPs), brain-derived neurotrophic factor (BDNF), epidermal growth factor (EGF), erythropoietin (EPO), fibroblast growth factor (FGF), fetal bovine somatotrophin (FBS), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin-like growth factor (IGF), keratinocyte growth factor (KGF), migration-stimulating factor (MSF), myostatin (GDF-8), nerve growth factor (NGF) and other neurotrophins, platelet-derived growth factor (PDGF), thrombopoietin (TPO), T-cell growth factor (TCGF), transforming growth factor alpha (TGF-α), transforming growth factor beta (TGF-β), tumor necrosis factor-alpha (TNF-α), vascular endothelial growth factor (VEGF), Wnt signaling pathway, placental growth factor (PGF), IL-1-Cofactor for IL-3 and IL-6, IL-2-T-cell growth factor, IL-3, IL-4, IL-5, IL-6, IL-7, and Renalase—RNLS—Anti-apoptotic survival factor.

In some embodiments, the therapeutic agent is a cell adhesion factor. Examples of cell adhesion factors include cadherins, immunoglobulin superfamily (Ig) CAMs, integrins, selectins. In some embodiments, the cell adhesion factor is an RGD peptide.

In some embodiments, the medicinal agent is water soluble.

### Coating and Adhesive Formation

Coatings may be produced by applying a dope solution onto a substrate and allowing the water and any volatile additives to evaporate.

Coatings prepared by the techniques disclosed herein can vary in their dimensions. Coating thicknesses can vary from 0.5 μm to 50 μm. In some embodiments, the coating thickness is from 1 to 25 μm. In some embodiments, the coating thickness is from 1 to 10 μm. In some embodiments, the coating thickness is from 1 to 25 μm.

Surfaces may be spray coated. The spray coatings can be applied in layers by applying a first coating followed by a period of drying. Following sufficient drying, second and subsequent coatings may be applied thereby creating layers of coatings. Optionally, between spray coatings, a different material such as medicinal agent may be added to a coating surface before another layer of recombinant spider silk solution is added. In some embodiments, the initial coating layer applied to a substrate surface is limited to a thin base layer to avoid beading or running on the surface. Subsequent layers added can be thicker.

Surfaces may also be dip coated. Dip coatings are prepared by taking the substrate or substrate surface and immersing it in an aqueous solution of solubilized recombinant spider silk. Once immersed, the substrate is removed to dry. Multiple layers of recombinant spider silk may be made to a surface by repeatedly dipping the surface or existing layer into the aqueous solution of solubilized recombinant spider silk followed by a brief drying period. In some embodiments, in between dip coatings, a different material such as a medicinal agent may be added to a coating surface before another layer of recombinant spider silk is added.

A combination of spray and dip coatings may also be applied. In one embodiment, an initial spray coated layer is applied to a substrate surface. Additional dip coatings may be applied to the initial spray coating. In such embodiments, surfaces treated with one or more spray coatings prior to dip coatings help to create an even coat and reduce beading and running. This combination of techniques also leads to better attachment for thicker coatings.

In addition, the techniques used here permit the preparation of medical devices that have coatings that facilitate hydrophobic substrates and, therefore, better biocompatibility and greater coating bond strengths. Also, adhesive strengths of the adhesives disclosed herein are superior to conventional adhesives that outperform conventional adhesion techniques depending on the substrate, silk type, and preparation method.

The solubilization process allows for coatings to be functionalized and tailored for specific purposes and functions. Functionalized coatings can include compounds such as therapeutic agents that are released on a predictable timescale from the recombinant spider silk coating. Functionally active coatings can prevent microbial growth and proliferation for short and longer periods such as a two week period. As discussed in the examples below, coatings have been functionalized with heparin, kanamycin, gentamicin, tetracycline, ampicillin, chloramphenicol, dexamethasone, azoles, and experimental antifungal compounds.

The following examples are illustrative only and are not intended to limit the disclosure in any way.

### Examples

Analogs of Nephila clavipes MaSp1 and MaSp2 (rMaSp1 and r MaSp2) proteins were expressed in the milk of transgenic goats and then purified through a process of tangential flow filtration, precipitation, washing, and lyophilization. (See Jones, Justin A., et al. “More Than Just Fibers: An Aqueous Method for the Production of Innovative Recombinant Spider Silk Protein Materials.” Biomacromolecules 16.4 (2015): 1418-1425.)

Analogs of Nephila clavipes MaSp1 and MaSp2 as well as chimeric proteins FLYS and FLAS (containing repetitive GPGGX motifs from N. Clavipes flagelliform silks and strength motifs polyAAA from major ampullate silks for FLYS. The FLAS constructs contain the elastic GPGGX with X being filled by A and then the same strength motifs previously described. See Teule, Florence, et al. “A protocol for the production of recombinant spider silk-like proteins for artificial fiber spinning.” Nature protocols 4.3 (2009): 341-355.

The previously published method for molecular design, fermentation, and purification of chimeric constructs FLYS and FLAS expressed in E. coli was used. Due to large volumes and spider silk's tendency to self-assemble under shear stress, ammonium sulfate precipitation and subsequent washing were used for the elution extraction. Precipitation and extraction of the protein was performed with a 3M solution of ammonium sulfate and 1/16 volume of isopropanol. This solution was allowed to mix overnight and the protein was extracted from the IPA phase. The product was washed with distilled water until conductivity is less than 10 μS/cm, and then the protein was collected and lyophilized. (See Teule, Florence, et al. “A protocol for the production of recombinant spider silk-like proteins for artificial fiber spinning.” Nature protocols 4.3 (2009): 341-355.)

A specified amount of purified recombinant spider silk proteins was placed in a glass vial (Wheaton) with the appropriate amount of distilled water (NanoPure Thermo Fischer) to create a dope of the desired concentration. This mixture was then sonicated (Q700, QSonica, LLC) at a power setting of W for one minute. The vial was sealed and solubilization of the rSSps occurred by heating the mixture in a 700 W Magic Chef microwave in 5 second intervals. Heating intervals were repeated until the mixture reached a temperature greater than or equal to 130° C., which was determined from cap temperature using a Fluke 561 hand-held infrared thermometer. The clarity of the solution was simultaneously monitored to determine when complete solubilization was achieved. Finally the dope was transferred into 1.5 mL eppendorf tubes and centrifuged at 18,000 rcf for 15 seconds to remove any particulates. All dope solutions were prepared using this standard technique.

Coatings were formed using a 6% (w/v) 80/20 rMaSp1/MaSp2 in distilled water. Various thicknesses and layers were developed by applying additional silk solution and building up the layers.

A G233 Master Airbrush with a 0.5 mm needle and nozzle connected to a TC-60 Master Airbrush compressor was used for coating applications. A layer of rSSP was applied from 50 cm away for 15 seconds and then allowed to dry for 3 minutes. Sprays were repeated until a desired thickness was achieved.

For dip coatings, a substrate was fully immersed in rSSp dope and allowed to dry. Multiple layers were obtained by repeated dip coatings to achieve higher amounts of silk and/or additives.

A combination of initially spraying and drying a layer of solvated rSSp dope onto the surface before dipping helped create an even coat and reduce beading and running. This combination of techniques also leads to better attachment for thicker coatings.

After formation centrifugation of 80/20 6% (w/v) dope, a 0.2 M ammonium bicarbonate solution was added to some dopes in a 1:1 ratio and mixed to form a final 3% (w/v) and 0.1 M ammonium bicarbonate solution. If bubbles were induced, centrifugation was used for removal of suspended gasses. This resulting mixture was then pipetted and spread onto the substrate surface and placed in a 35° C. oven for 24 hours.

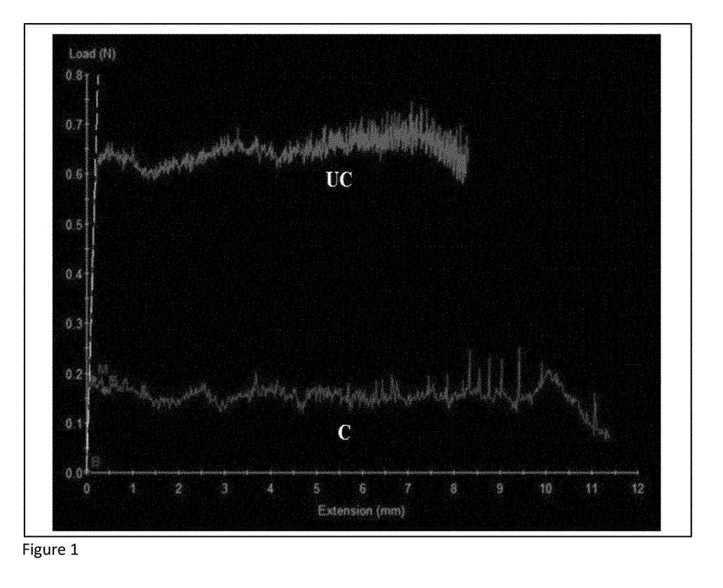

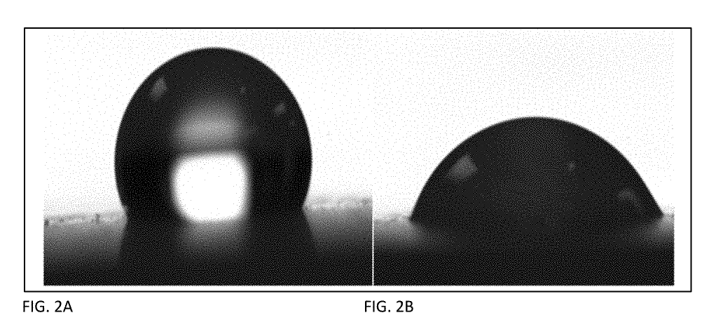

Various characterization tests were performed on coated substrates to determine the surface properties of the coatings. Coefficient of friction tests were performed on silicone and polycarbonate substrates that were coated and uncoated. All samples were pulled at a constant rate of 10 mm/min, a normal force of 3 N was applied, and gauge length of 20 mm was used. The data were then analyzed with Microsoft Excel and the static and kinetic coefficients were determined from these data. See FIG. 1. Contact angle measurements were also conducted on the similarly prepared samples but the addition of various post-treatments using a goniometer and software to analyze the contact angle. The samples were brought into focus and then 10 μL of distilled water was applied, and the image was captured and the angles were calculated. See FIG. 2.

### Antimicrobial Functionalization

The silicone, stainless steel, and catheters samples that were used for these experiments were first sterilized prior to preparation and stored accordingly. The spray solution was prepared as previously described and allowed to cool below 50° C. before the appropriate additives were added. Because of the solid nature of the coatings, the concentrations for antimicrobials were usually doubled (50 μg/mL 100 m/mL) to allow for appropriate diffusion and activity of the additives.

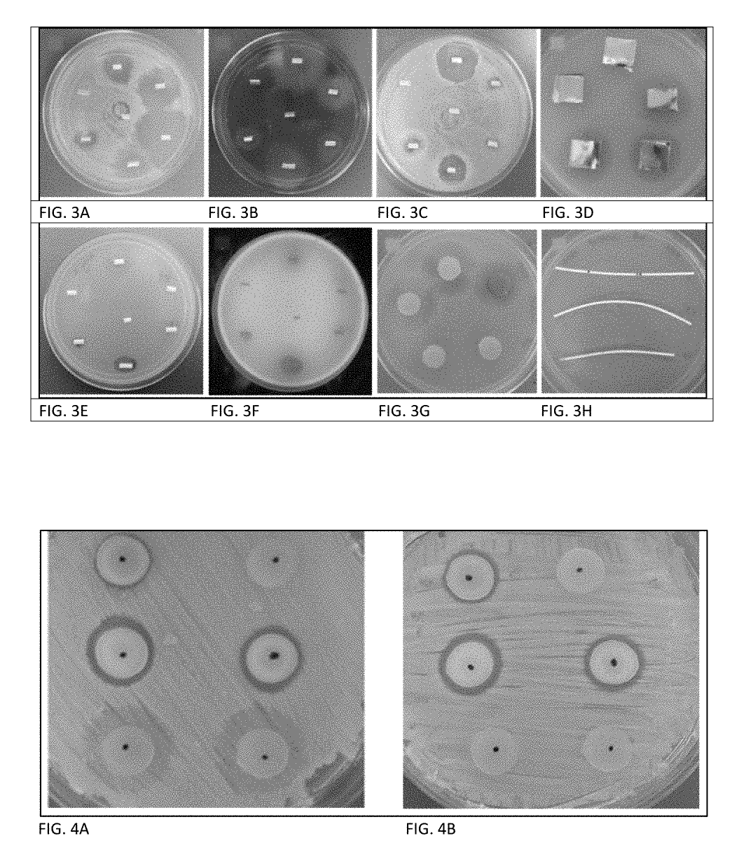

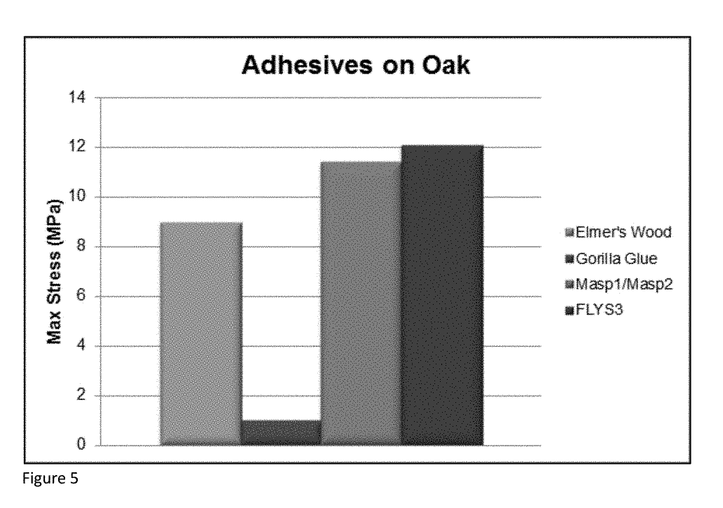

Base layers were sprayed onto the test surfaces using an 80/20 (rMaSp1/MaSp2) 6% (w/v) solution with the functionalizing compounds. Each base layer was dried for 15 minutes at 25° C. Next, three dip coatings were applied, using the functionalized dope, with three minutes of drying in between each application. These samples were then fully dried overnight, and overnight liquid suspension cultures of the microbes were started. After twelve to sixteen hours of growth, the microbes were streaked onto appropriate plates and dried. Loaded samples were placed onto the plates, and these were then placed in a 37° C. incubator for 24 hours. The plates were then removed, and the inhibition zones were measured and recorded. The plates were then placed back in the incubator and checked daily until the zones were overgrown. See FIGS. 3 and 4.

### Antithrombotic Functionalization

Blood was obtained and treated with 0.3% sodium citrate to prevent clotting. This blood was refrigerated and stored for a maximum of three days and then placed in an incubator for one hour prior to use in this study. Catheters, stainless steel, or titanium segments were cut and a base layer was sprayed using an 80/20 (rMaSp1/MaSp2) 6% (w/v) solution. After drying, three layers of a 80/20 (rMaSp1/MaSp2) 6% (w/v) solution with 1% (w/v) heparin were sprayed onto samples. Then an initial sample dry weight was obtained. For each study, nine samples were prepared: three controls (no spider silk), three spider silk controls sprayed with 80/20 (rMaSp1/rMaSp2) 6% (w/v) solution, and three experimental spider silk samples loaded with 1% (w/v) of heparin. These samples were then placed into a 15 mL conical tube containing 10 mL of the blood/sodium citrate and rotated in a 37° C. incubator for 1 hour. After incubation, the samples were removed from the incubator and calcium chloride was added to a final concentration of 50 mM to the conicals. The tubes were then rotated until signs of clotting were evident. Clotting times were recorded for each conical and the catheters were quickly removed from the clotted blood, washed with distilled water, and then placed in a petri dish. The catheters were allowed to dry in a 35° C. oven for two days before final weights were obtained and analyzed. Results were as follows:

Dry Mass Increase (mg)

| | | | Spider |
|---|---|---|---|
| Sample | No Coating | Spider Silk | Silk + Heparin |
| Polyurethane | 479% | 153% | 15.8% |
| Silicone | 429% | 233.8% | 3.8% |
| Stainless Steel | 2.5% | 1.9% | 0.08% |

Adhesive Formulation

Adhesives were created using a 50/50 blend of rMaSp1/MaSp2, in either a 6%, 12%, or 20% (w/v) in distilled water. FLYS constructs were dissolved in a 40:60 mixture of isopropyl alcohol:distilled water. The combination of the adhesive solution and the previously described bicarbonate additive is also possible during this stage and frequently used for better properties.

A base coating of silk on the surface to be adhered was created using the spray technique described above. Layers are repeated until a visible coating is apparent on the surface. The visible coating is then allowed to dry at 25° C. for 15 minutes. After drying, 80 μL/cm² are pipetted and spread onto one side of surface to be adhered. The two pieces were then applied together for 5 seconds and placed in an oven at 35° C. oven for 24 hours.

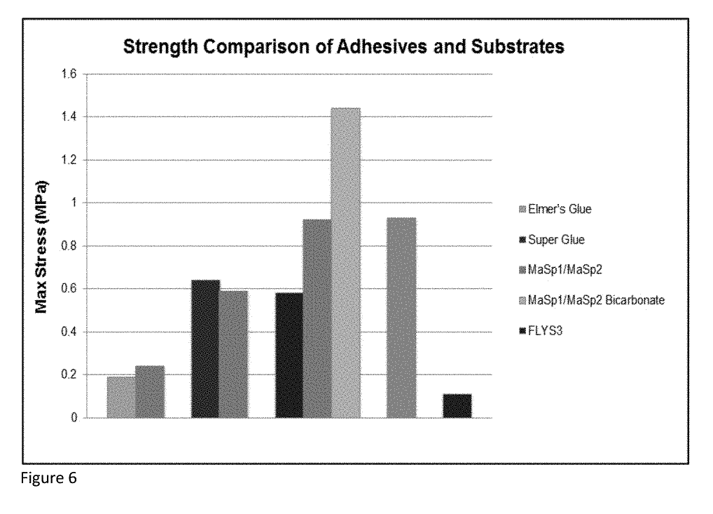

Wood and metal samples were tested in a Tinius Olsen H50KS 50 kN using Tinius Olsen utility software in the utility mode at 10 mm/min with data acquisition at 8 Hz. The silicone samples were tested on a 50 N MTS Synergie 100 at 1.3 mm/min, with readings taken at 30 Hz using TestWorks4, 2001 to process the information. All other substrates and materials were tested on a 250 N MTS Tytron 250 at 1.3 mm/min with data collected at using Testworks4. All testing units were prepared in a manner that would allow later shear tests to be performed. The data were then processed and analyzed with Microsoft Excel to determine the stress, strain, toughness, and moduli of the adhesives. See FIGS. 5 and 6.

| | Maximum | Maximum | |
|---|---|---|---|
| | Stress | Strain | Toughness |
| Adhesive - Substrate | (MPa) | (%) | (kJ/m³) |
| FlYS₃ - Wood | 13.86 | 31 | 2180 |
| MaSp1/MaSp2 - Wood | 15.35 | 26 | 2940 |
| Gorilla Glue - Wood | 1.98 | 3.7 | 49 |
| Elmer's Wood Glue - Wood | 10.8 | 12 | 664 |
| FlYS₃ - Polycarbonate | 0.62 | 11 | 21 |
| MaSp1/MaSp2 - Polycarbonate | 0.74 | 9 | 31 |
| MaSp1/MaSp2 - Polypropylene | 0.97 | 18 | 225 |
| MaSp1/MaSp2 - Silicone | 0.06 | 35 | 12 |
| Elmer's - Silicone | 0.02 | 6.1 | 0.7 |

It will be appreciated that variations of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A method of coating a medical device, comprising:
solubilizing one or more recombinant spider silk proteins in an aqueous solution;
providing a medical device having a substrate surface;
applying the solubilized one or more recombinant spider silk proteins in the aqueous solution to the substrate surface;
drying the device surface.

2. The method of claim 1, wherein the solubilizing step further comprises:
mixing the one or more recombinant spider silk proteins with water to form a mixture in a sealed container;
heating the mixture.

3. The method of claim 2, wherein the heating is performed with microwave irradiation.

4. The method of claim 1, wherein the application of the solubilized recombinant spider silk is by an aerosolizing sprayer.

5. The method of claim 1, wherein the application of the solubilized recombinant spider silk is performed by dipping the substrate surface in the aqueous solution.

6. The method of claim 5, further comprising applying a base-layer coating to the substrate surface prior to applying additional layers of the solubilized recombinant spider silk.

7. The method of claim 1, further comprising adding an additive to the aqueous solution.

8. The method of claim 7, wherein the additive is a bicarbonate salt.

9. The method of claim 7, wherein the additive decreases drying time.

10. The method of claim 9, wherein the additive is an organic alcohol or an organic acid.

11. The method of claim 1, wherein the substrate surface is selected from a cellulosic polymer, a silicone polymer, a plastic polymer, and a metal.

12. The method of claim 1, wherein the one or more recombinant spider silk proteins is selected from the group consisting of: M4, M5, MaSp1, an MaSp1 analogue, MaSp2, an MaSp2 analogue, FLYS, FLAS, AcSp (aciniform silk protein), AgSp2 (aggregate gland silk protein 2), and combinations thereof.

13. The method of claim 1, further comprising adding a medicinal agent.

14. The method of claim 13, wherein the medicinal agent is selected from an antimicrobial agent, an antifungal agent, and antibacterial agent an anti-clotting agent, heparin, and a therapeutic agent.

15. A medical device prepared from the method of claim 1.

16. The medical device of claim 15, wherein the medical device is selected from a catheter, a splint, a bandage, a drain tube, and an implant.

17. A method of adhering two objects, comprising:
solubilizing one or more recombinant spider silk proteins in an aqueous solution;
providing a first object having a substrate surface;
applying the solubilized one or more recombinant spider silk proteins in the aqueous solution to the substrate surface;
providing a second objecting having a target surface;
contacting the substrate surface and the target surface to adhere the first and second objects together.

18. The method of claim 17, further comprising removing water from between the substrate and target surfaces.

19. The method of claim 17, wherein the target surface of the second object is selected from a cellulosic polymer, a silicone polymer, a plastic polymer, and a metal.
